(19) United States
(12) Patent Application Publication
(10) Pub. No.: US 2015/0275237 A1
(43) Pub. Date: Oct. 1, 2015

(54) PROCESSES FOR CO-PRODUCING XYLITOL WITH ETHANOL OR OTHER FERMENTATION PRODUCTS

(71) Applicant: API Intellectual Property Holdings, LLC, Atlanta, GA (US)

(72) Inventors: Theodora RETSINA, Atlanta, GA (US); Vesa PYLKKANEN, Atlanta, GA (US)

(21) Appl. No.: 14/663,437

(22) Filed: Mar. 19, 2015

Related U.S. Application Data
(60) Provisional application No. 61/970,250, filed on Mar. 25, 2014.

(51) Int. Cl. C12P 7/18; C12P 7/14

(52) U.S. Cl. CPC C12P 7/18; C12P 7/14; C12P 2201/00; C12P 2203/00

(57) ABSTRACT

What is disclosed is a biorefining process to co-produce xylitol with ethanol or other products. In some variations, a process for producing ethanol and xylitol from lignocellulosic biomass, comprises: extracting hemicelluloses from lignocellulosic biomass, wherein the hemicelluloses include xylose oligomers and other sugar oligomers; hydrolyzing the xylose oligomers and the other sugar oligomers, using an acid catalyst or enzymes, to generate xylose and other sugar monomers, respectively; fermenting the other sugar monomers to ethanol using a suitable ethanol-producing microorganism; removing at least some of the ethanol (to increase concentration of xylose); fermenting the xylose to xylitol using a suitable xylitol-producing microorganism; and recovering the xylitol at high concentration.

### PRIORITY DATA

This patent application is a non-provisional patent application claiming priority to U.S. Provisional Patent App. No. 61/970,250, filed on Mar. 25, 2014, which is hereby incorporated by reference herein.

### FIELD

The present invention generally relates to biorefining processes for converting biomass into xylitol and other products.

### BACKGROUND

Lignocellulosic biomass is the most abundant renewable material on the planet and has long been recognized as a potential feedstock for producing chemicals, fuels, and materials. Lignocellulosic biomass normally comprises primarily cellulose, hemicellulose, and lignin. Cellulose and hemicellulose are natural polymers of sugars, and lignin is an aromatic/aliphatic hydrocarbon polymer reinforcing the entire biomass network.

Xylitol is a five-carbon sugar alcohol that can be found in nature in small quantities. It has attracted global attention because of its sweetening power similar to that of sucrose, but provides much fewer calories. Xylitol is also known to be metabolized through insulin-independent pathways in human body and therefore it can be used as sugar substitute for diabetics.

Currently, xylitol is industrially produced by chemical hydrogenation of xylose-containing hemicellulosic hydrolysate in the presence of a metal catalyst under high temperature and pressure However, this chemical process is costly and energy-intensive; in addition, there is also a need for a complex purification and separation process. In order to produce xylitol in a more environmental-friendly manner, research has been conducted on alternative strategies that utilize microorganisms for conversion of xylose-to-xylitol from hemicelluloses (Prakasham et al., “Current trends in biotechnological production of xylitol and future prospects,” Curr. Trends Biotechnol. Pharm. 3, 8-36, 2009; Granström, et al., “A rare sugar xylitol. Part I: the biochemistry and biosynthesis of xylitol,” Appl. Microbiol. Biotechnol. 74, 277-281, 2007).

Improved processes to produce and recover xylitol are needed commercially.

### SUMMARY

The present invention addresses the aforementioned needs in the art.

In some variations, the invention provides a process for producing ethanol and xylitol from lignocellulosic biomass, the process comprising:
(a) extracting hemicelluloses from a feedstock comprising lignocellulosic biomass, wherein the hemicelluloses include xylose oligomers and other sugar oligomers;
(b) hydrolyzing the xylose oligomers and the other sugar oligomers, using an acid catalyst or enzymes, to generate xylose and other sugar monomers, respectively;
(c) fermenting the other sugar monomers to ethanol using a suitable ethanol-producing microorganism;
(d) optionally removing at least some of the ethanol;
(e) fermenting the xylose to xylitol using a suitable xylitol-producing microorganism;
(f) recovering the ethanol, derived from step (c) and/or, if conducted, step (d); and
(g) recovering the xylitol derived from step (e).

In some variations, the invention provides a process for producing xylitol and a C₆ fermentation product from lignocellulosic biomass, the process comprising:
(a) extracting hemicelluloses from a feedstock comprising lignocellulosic biomass, wherein the hemicelluloses include xylose oligomers and C₆ sugar oligomers;
(b) hydrolyzing the xylose oligomers and the C₆ sugar oligomers, using an acid catalyst or enzymes, to generate xylose and C₆ sugar monomers, respectively;
(c) fermenting the C₆ sugar monomers to a C₆ fermentation product using a suitable C₆-fermenting microorganism;
(d) optionally removing at least some of the C₆ fermentation product;
(e) fermenting the xylose to xylitol using a suitable xylitol-producing microorganism;
(f) recovering the C6 fermentation product, derived from step (c) and/or, if conducted, step (d); and
(g) recovering the xylitol derived from step (e).

In some embodiments, step (a) utilizes steam and/or hot water, optionally with an extraction catalyst. In these or other embodiments, step (a) utilizes an extraction catalyst (such as sulfur dioxide), a solvent for lignin (such as ethanol), and water.

In some embodiments, step (b) utilizes a sulfur-containing acid catalyst. In certain embodiments, step (b) utilizes an acid catalyst derived from step (a), such as acetic acid or lignosulfonic acids.

In some embodiments, step (d) is conducted. Steps (c) and (e) may be conducted simultaneously or sequentially (or both). Steps (f) and (g) may be conducted in the same fermentor and/or in different fermentors. That is, xylitol and ethanol (or another C₆ fermentation product) may be recovered from a single fermentor and may be produced in and recovered from distinct fermentors.

The process further comprises, in preferred embodiments, removing lignin derived from the lignocellulosic biomass.

Other variations provide a process for producing ethanol and xylitol from lignocellulosic biomass, the process comprising:
(a) extracting hemicelluloses from a feedstock comprising lignocellulosic biomass, wherein the hemicelluloses include xylose oligomers and other sugar oligomers;
(b) hydrolyzing the xylose oligomers and the other sugar oligomers, using an acid catalyst or enzymes, to generate xylose and other sugar monomers, respectively;
(c) co-fermenting the xylose to xylitol and the other sugar monomers to ethanol, using a suitable microorganism or combination of microorganisms;
(d) continuously removing the ethanol to increase concentration of the xylitol; and
(e) recovering the xylitol.

Other variations provide a process for producing xylitol and a C₆ fermentation product from lignocellulosic biomass, the process comprising:
(a) extracting hemicelluloses from a feedstock comprising lignocellulosic biomass, wherein the hemicelluloses include xylose oligomers and C₆ sugar oligomers;
(b) hydrolyzing the xylose oligomers and the C₆ sugar oligomers, using an acid catalyst or enzymes, to generate xylose and C₆ sugar monomers, respectively;
(c) co-fermenting the xylose to xylitol and the C₆ sugar monomers to a C₆ fermentation product, using a suitable microorganism or combination of microorganisms;
(d) continuously removing the a C₆ fermentation product to increase concentration of the xylitol; and
(e) recovering the xylitol.

In any of these variations or embodiments, the process may further include recovering or generating a cellulose-rich material from the lignocellulosic biomass. The cellulose-rich material may be recovered following an initial fractionation of feedstock, for example. Alternatively, or additionally, the cellulose-rich material may remain in a process stream along with extracted hemicelluloses and may remain during oligomer hydrolysis and fermentation, in some embodiments. The cellulose-rich material may be selected from the group consisting of market pulp, dissolving pulp, fluff pulp, purified cellulose, functionalized cellulose, nanocellulose, paper, boxes, boards, pellets, and combinations thereof.

Another embodiment provides a process for producing xylitol and a C₆ fermentation product from lignocellulosic biomass, the process comprising:
(a) extracting hemicelluloses from a feedstock comprising lignocellulosic biomass, wherein the hemicelluloses include xylose oligomers and C₆ sugar oligomers;
(b) fermenting the xylose oligomers to xylitol using a suitable microorganism capable of converting xylose oligomers directly into xylitol;
(c) hydrolyzing the C₆ sugar oligomers, using an acid catalyst or enzymes, to generate C₆ sugar monomers;
(d) fermenting the C₆ sugar monomers to a C₆ fermentation product using a suitable C₆-fermenting microorganism, wherein at least some of the C₆ fermentation product is optionally removed;
(e) recovering the C₆ fermentation product, derived from step (d); and
(f) recovering the xylitol derived from step (b).

Another embodiment provides a composition comprising xylitol, the composition produced by a process as disclosed.

Some variations provide a process for producing xylitol and another fermentation product from lignocellulosic biomass, the process comprising:
(a) extracting hemicelluloses from a feedstock comprising lignocellulosic biomass, wherein the hemicelluloses include xylose oligomers and other sugar oligomers;
(b) hydrolyzing the xylose oligomers and the other sugar oligomers, to generate xylose and other sugar monomers, respectively, using an acid catalyst or enzymes;
(c) fermenting the other sugar monomers to a first fermentation product, using a suitable first microorganism;
(d) optionally removing at least some of the first fermentation product;
(e) fermenting the xylose to a second fermentation product comprising xylitol, using a suitable second microorganism;
(f) recovering the first fermentation product, derived from step (c) and/or, if conducted, step (d); and
(g) recovering the xylitol derived from step (e).

In some embodiments, the other sugar oligomers include C₆ sugars. In some embodiments, the first fermentation product comprises ethanol.

In some embodiments, wherein step (a) utilizes steam and/or hot water, optionally with an extraction catalyst. In these or other embodiments, step (a) utilizes an extraction catalyst, a solvent for lignin, and water. Step (b) may utilize a sulfur-containing acid catalyst, which optionally is derived from step (a).

Steps (c) and (e) may be conducted simultaneously, sequentially, or both of these.

The process may further include removing lignin derived from the lignocellulosic biomass. In some embodiments, the process further comprises recovering or generating a cellulose-rich material from the lignocellulosic biomass, wherein the cellulose-rich material is selected from the group consisting of market pulp, dissolving pulp, fluff pulp, purified cellulose, functionalized cellulose, nanocellulose, paper, boxes, boards, pellets, and combinations thereof.

Other variations provide a process for producing xylitol and another fermentation product from lignocellulosic biomass, the process comprising:
(a) extracting hemicelluloses from a feedstock comprising lignocellulosic biomass, wherein the hemicelluloses include xylose oligomers and other sugar oligomers;
(b) hydrolyzing the xylose oligomers and the other sugar oligomers, to generate xylose and other sugar monomers, respectively, using an acid catalyst or enzymes;
(c) co-fermenting the xylose to a first fermentation product comprising xylitol and the other sugar monomers to a second fermentation product, using a suitable microorganism or combination of microorganisms;
(d) continuously removing the second fermentation product to increase concentration of the xylitol; and
(e) recovering the xylitol.

### DETAILED DESCRIPTION OF SOME EMBODIMENTS

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing parameters, reaction conditions, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of.”

The present invention, in some variations, is premised on the realization that an important aspect of commercial production of xylitol is the ability to achieve high concentration of its precursor, xylose. By first fermenting other sugars to ethanol or other products, or by co-fermenting a sugar solution under conditions in which the other sugars (besides xylose) are fermented faster than xylose, the xylose concentration can be enriched. When such xylose is then fermented to xylitol, it will be in relatively high concentration for more efficient separation and purification. For example if half the initial sugars are xylose and half are C₆ sugars, completely fermenting the C₆ sugars and optionally removing the products, means that the xylose purity doubles. Even accounting from some non-fermented C₅ or C₆ sugars, the xylose purity may increase from ˜40% to ˜80% of the sugars present, for example.

Certain exemplary embodiments of the invention will now be described. These embodiments are not intended to limit the scope of the invention as claimed. The order of steps may be varied, some steps may be omitted, and/or other steps may be added. Reference herein to first step, second step, etc. is for illustration purposes only.

Some variations provide a process for producing xylitol and another fermentation product from lignocellulosic biomass, the process comprising:
(a) extracting hemicelluloses from a feedstock comprising lignocellulosic biomass, wherein the hemicelluloses include xylose oligomers and other sugar oligomers;
(b) hydrolyzing the xylose oligomers and the other sugar oligomers, to generate xylose and other sugar monomers, respectively, using an acid catalyst or enzymes;
(c) fermenting the other sugar monomers to a first fermentation product, using a suitable first microorganism;
(d) optionally removing at least some of the first fermentation product;
(e) fermenting the xylose to a second fermentation product comprising xylitol, using a suitable second microorganism;
(f) recovering the first fermentation product, derived from step (c) and/or, if conducted, step (d); and
(g) recovering the xylitol derived from step (e).

In some embodiments, the other sugar oligomers include C₅ and/or C₆ sugars. In some embodiments, the first fermentation product comprises ethanol or another alcohol, such as n-butanol.

In some embodiments, wherein step (a) utilizes steam and/or hot water, optionally with an extraction catalyst. In these or other embodiments, step (a) utilizes an extraction catalyst, a solvent for lignin, and water. Step (b) may utilize a sulfur-containing acid catalyst, which optionally is derived from step (a).

Steps (c) and (e) may be conducted simultaneously, sequentially, or both of these.

The process may further include removing lignin derived from the lignocellulosic biomass. In some embodiments, the process further comprises recovering or generating a cellulose-rich material from the lignocellulosic biomass, wherein the cellulose-rich material is selected from the group consisting of market pulp, dissolving pulp, fluff pulp, purified cellulose, functionalized cellulose, nanocellulose, paper, boxes, boards, pellets, and combinations thereof.

Other variations provide a process for producing xylitol and another fermentation product from lignocellulosic biomass, the process comprising:
(a) extracting hemicelluloses from a feedstock comprising lignocellulosic biomass, wherein the hemicelluloses include xylose oligomers and other sugar oligomers;
(b) hydrolyzing the xylose oligomers and the other sugar oligomers, to generate xylose and other sugar monomers, respectively, using an acid catalyst or enzymes;
(c) co-fermenting the xylose to a first fermentation product comprising xylitol and the other sugar monomers to a second fermentation product, using a suitable microorganism or combination of microorganisms;
(d) continuously removing the second fermentation product to increase concentration of the xylitol; and
(e) recovering the xylitol.

In some variations, the invention provides a process for producing ethanol and xylitol from lignocellulosic biomass, the process comprising:
(a) extracting hemicelluloses from a feedstock comprising lignocellulosic biomass, wherein the hemicelluloses include xylose oligomers and other sugar oligomers;
(b) hydrolyzing the xylose oligomers and the other sugar oligomers, using an acid catalyst or enzymes, to generate xylose and other sugar monomers, respectively;
(c) fermenting the other sugar monomers to ethanol using a suitable ethanol-producing microorganism;
(d) optionally removing at least some of the ethanol;
(e) fermenting the xylose to xylitol using a suitable xylitol-producing microorganism;
(f) recovering the ethanol, derived from step (c) and/or, if conducted, step (d); and
(g) recovering the xylitol derived from step (e).

In some variations, the invention provides a process for producing xylitol and a C₆ fermentation product from lignocellulosic biomass, the process comprising:
(a) extracting hemicelluloses from a feedstock comprising lignocellulosic biomass, wherein the hemicelluloses include xylose oligomers and C₆ sugar oligomers;
(b) hydrolyzing the xylose oligomers and the C₆ sugar oligomers, using an acid catalyst or enzymes, to generate xylose and C₆ sugar monomers, respectively;
(c) fermenting the C₆ sugar monomers to a C₆ fermentation product using a suitable C₆-fermenting microorganism;
(d) optionally removing at least some of the C₆ fermentation product;
(e) fermenting the xylose to xylitol using a suitable xylitol-producing microorganism;
(f) recovering the C₆ fermentation product, derived from step (c) and/or, if conducted, step (d); and
(g) recovering the xylitol derived from step (e).

In some embodiments, step (a) utilizes steam and/or hot water, optionally with an extraction catalyst. In these or other embodiments, step (a) utilizes an extraction catalyst (such as sulfur dioxide), a solvent for lignin (such as ethanol), and water.

In some embodiments, step (b) utilizes a sulfur-containing acid catalyst. In certain embodiments, step (b) utilizes an acid catalyst derived from step (a), such as acetic acid or lignosulfonic acids.

In some embodiments, step (d) is conducted. Steps (c) and (e) may be conducted simultaneously or sequentially (or both). Steps (f) and (g) may be conducted in the same fermentor and/or in different fermentors. That is, xylitol and ethanol (or another C₆ fermentation product) may be recovered from a single fermentor and may be produced in and recovered from distinct fermentors.

Suitable C₆-fermenting microorganisms and ethanol-producing microorganisms include a wide variety of well-known yeasts, bacteria, and algae. Suitable xylitol-producing microorganisms are also known in the art. For example, see Huang, et al., “Development of a yeast strain for xylitol production without hydrolysate detoxification as part of the integration of co-product generation within the lignocellulosic ethanol process” Bioresource Technology 102 (2011) 3322-3329; Latif et al., “Production of ethanol and xylitol from corn cobs by yeasts,” Bioresource Technology 77 (2001) 57-63; and Rao et al., “Xylitol production by Candida sp.: parameter optimization using Taguchi approach,” Process Biochemistry 39 (2004) 951-956, each of which is hereby incorporated by reference.

The process further comprises, in preferred embodiments, removing lignin derived from the lignocellulosic biomass. The lignin may be removed during hemicellulose hydrolysis, or following such step. Lignin removal may be integrated with removal of ethanol or other C₆ fermentation product, to increase purity of xylitol.

The biomass feedstock may be selected from hardwoods, softwoods, forest residues, industrial wastes, pulp and paper wastes, consumer wastes, or combinations thereof. Some embodiments utilize agricultural residues, which include lignocellulosic biomass associated with food crops, annual grasses, energy crops, or other annually renewable feedstocks. Exemplary agricultural residues include, but are not limited to, corn stover, corn fiber, wheat straw, sugarcane bagasse, sugarcane straw, rice straw, oat straw, barley straw, miscanthus, energy cane straw/residue, or combinations thereof.

As used herein, “lignocellulosic biomass” means any material containing cellulose and lignin. Lignocellulosic biomass may also contain hemicellulose. Mixtures of one or more types of biomass can be used. In some embodiments, the biomass feedstock comprises both a lignocellulosic component (such as one described above) in addition to a sucrose-containing component (e.g., sugarcane or energy cane) and/or a starch component (e.g., corn, wheat, rice, etc.).

Various moisture levels may be associated with the starting biomass. The biomass feedstock need not be, but may be, relatively dry. In general, the biomass is in the form of a particulate or chip, but particle size is not critical in this invention.

Other variations provide a process for producing ethanol and xylitol from lignocellulosic biomass, the process comprising:
(a) extracting hemicelluloses from a feedstock comprising lignocellulosic biomass, wherein the hemicelluloses include xylose oligomers and other sugar oligomers;
(b) hydrolyzing the xylose oligomers and the other sugar oligomers, using an acid catalyst or enzymes, to generate xylose and other sugar monomers, respectively;
(c) co-fermenting the xylose to xylitol and the other sugar monomers to ethanol, using a suitable microorganism or combination of microorganisms;
(d) continuously removing the ethanol to increase concentration of the xylitol; and
(e) recovering the xylitol.

Other variations provide a process for producing xylitol and a C₆ fermentation product from lignocellulosic biomass, the process comprising:
(a) extracting hemicelluloses from a feedstock comprising lignocellulosic biomass, wherein the hemicelluloses include xylose oligomers and C₆ sugar oligomers;
(b) hydrolyzing the xylose oligomers and the C₆ sugar oligomers, using an acid catalyst or enzymes, to generate xylose and C₆ sugar monomers, respectively;
(c) co-fermenting the xylose to xylitol and the C₆ sugar monomers to a C₆ fermentation product, using a suitable microorganism or combination of microorganisms;
(d) continuously removing the a C₆ fermentation product to increase concentration of the xylitol; and
(e) recovering the xylitol.

In any of these variations or embodiments, the process may further include recovering or generating a cellulose-rich material from the lignocellulosic biomass. The cellulose-rich material may be recovered following an initial fractionation of feedstock, for example. Alternatively, or additionally, the cellulose-rich material may remain in a process stream along with extracted hemicelluloses and may remain during oligomer hydrolysis and fermentation, in some embodiments. The cellulose-rich material may be selected from the group consisting of market pulp, dissolving pulp, fluff pulp, purified cellulose, functionalized cellulose, nanocellulose, paper, boxes, boards, pellets, and combinations thereof.

Another embodiment provides a process for producing xylitol and a C₆ fermentation product from lignocellulosic biomass, the process comprising:
(a) extracting hemicelluloses from a feedstock comprising lignocellulosic biomass, wherein the hemicelluloses include xylose oligomers and C₆ sugar oligomers;
(b) fermenting the xylose oligomers to xylitol using a suitable microorganism capable of converting xylose oligomers directly into xylitol;
(c) hydrolyzing the C₆ sugar oligomers, using an acid catalyst or enzymes, to generate C₆ sugar monomers;
(d) fermenting the C₆ sugar monomers to a C₆ fermentation product using a suitable C₆-fermenting microorganism, wherein at least some of the C₆ fermentation product is optionally removed;
(e) recovering the C₆ fermentation product, derived from step (d); and
(f) recovering the xylitol derived from step (b).

Another embodiment provides a composition comprising xylitol, the composition produced by a process as disclosed.

Another embodiment provides apparatus configured for carrying out a process as disclosed.

In some embodiments, the cellulose-rich solids are utilized as pulp for production of a material (such as nanocellulose), pellet, or consumer product. Alternatively, or additionally, the cellulose-rich solids may be combusted to produce energy. The cellulose-rich solids may also be enzymatically hydrolyzed to produce glucose.

Reaction conditions and operation sequences may vary widely. In some embodiments, the process is a variation of the AVAP® process technology which is commonly owned with the assignee of this patent application. In some embodiments, the process is a variation of the Green Power+® process technology which is commonly owned with the assignee of this patent application.

Fermentable sugars are defined as hydrolysis products of cellulose, galactoglucomannan, glucomannan, arabinoglucuronoxylans, arabinogalactan, and glucuronoxylans into their respective short-chained oligomers and monomer products, i.e., glucose, mannose, galactose, xylose, and arabinose. The fermentable sugars may be recovered in purified form, as a sugar slurry or dry sugar solids, for example. Any known technique may be employed to recover a slurry of sugars or to dry the solution to produce dry sugar solids.

In some embodiments, the fermentable sugars are fermented to produce biochemicals or biofuels such as (but by no means limited to) ethanol, isopropanol, acetone, 1-butanol, isobutanol, lactic acid, succinic acid, or any other fermentation products. Some amount of the fermentation product may be a microorganism or enzymes, which may be recovered if desired.

Any stream generated by the disclosed processes may be partially or completed recovered, purified or further treated, analyzed (including on-line or off-line analysis), and/or marketed or sold.

Apparatus may be configured for carrying out the disclosed processes using chemical-engineering principles known in the art as well as principles disclosed in commonly owned patents and patent applications, cited above and incorporated by reference herein.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A process for producing xylitol and another fermentation product from lignocellulosic biomass, said process comprising:
(a) extracting hemicelluloses from a feedstock comprising lignocellulosic biomass, wherein said hemicelluloses include xylose oligomers and other sugar oligomers;
(b) hydrolyzing said xylose oligomers and said other sugar oligomers, to generate xylose and other sugar monomers, respectively, using an acid catalyst or enzymes;
(c) fermenting said other sugar monomers to a first fermentation product, using a suitable first microorganism;
(d) optionally removing at least some of said first fermentation product;
(e) fermenting said xylose to a second fermentation product comprising xylitol, using a suitable second microorganism;
(f) recovering said first fermentation product, derived from step (c) and/or, if conducted, step (d); and
(g) recovering said xylitol derived from step (e).

2. The process of claim 1, wherein said other sugar oligomers include C₆ sugars.

3. The process of claim 1, wherein said first fermentation product comprises ethanol.

4. The process of claim 1, wherein step (a) utilizes steam and/or hot water, optionally with an extraction catalyst.

5. The process of claim 1, wherein step (a) utilizes an extraction catalyst, a solvent for lignin, and water.

6. The process of claim 1, wherein step (b) utilizes a sulfur-containing acid catalyst.

7. The process of claim 6, wherein sulfur-containing acid catalyst is derived from step (a).

8. The process of claim 1, wherein steps (c) and (e) are conducted simultaneously.

9. The process of claim 1, wherein steps (c) and (e) are conducted sequentially.

10. The process of claim 1, said process further comprising removing lignin derived from said lignocellulosic biomass.

11. The process of claim 1, said process further comprising recovering or generating a cellulose-rich material from said lignocellulosic biomass, wherein said cellulose-rich material is selected from the group consisting of market pulp, dissolving pulp, fluff pulp, purified cellulose, functionalized cellulose, nanocellulose, paper, boxes, boards, pellets, and combinations thereof.

12. A process for producing xylitol and another fermentation product from lignocellulosic biomass, said process comprising:
(a) extracting hemicelluloses from a feedstock comprising lignocellulosic biomass, wherein said hemicelluloses include xylose oligomers and other sugar oligomers;
(b) hydrolyzing said xylose oligomers and said other sugar oligomers, to generate xylose and other sugar monomers, respectively, using an acid catalyst or enzymes;
(c) co-fermenting said xylose to a first fermentation product comprising xylitol and said other sugar monomers to a second fermentation product, using a suitable microorganism or combination of microorganisms;
(d) continuously removing said second fermentation product to increase concentration of said xylitol; and
(e) recovering said xylitol.

13. The process of claim 12, wherein said other sugar oligomers include C₆ sugars.

14. The process of claim 12, wherein said second fermentation product comprises ethanol.

15. The process of claim 12, wherein step (a) utilizes steam and/or hot water, optionally with an extraction catalyst.

16. The process of claim 12, wherein step (a) utilizes an extraction catalyst, a solvent for lignin, and water.

17. The process of claim 12, wherein step (b) utilizes a sulfur-containing acid catalyst.

18. The process of claim 17, wherein sulfur-containing acid catalyst is derived from step (a).

19. The process of claim 12, said process further comprising removing lignin derived from said lignocellulosic biomass.

20. The process of claim 12, said process further comprising recovering or generating a cellulose-rich material from said lignocellulosic biomass, wherein said cellulose-rich material is selected from the group consisting of market pulp, dissolving pulp, fluff pulp, purified cellulose, functionalized cellulose, nanocellulose, paper, boxes, boards, pellets, and combinations thereof.
